(19) United States
(12) Patent Application Publication
(10) Pub. No.: US 2003/0158128 A1
(43) Pub. Date: Aug. 21, 2003

(54) Treatment, composition and method using uracil against side-effects of chemotherapy

(76) Inventor: John P. Ford, Unadilla, NY (US)

Correspondence Address: Bernard Malina, Malina & Wolson, 60 East 42nd Street, New York, NY, 10165, US

(21) Appl. No.: 10/364,383

(22) Filed: Feb. 12, 2003

Related U.S. Application Data
(60) Provisional application No. 60/355,764, filed on Feb. 12, 2002.

(51) Int. Cl. A61K 31/7072; A61K 31/704; A61K 31/513; A61K 31/337

(52) U.S. Cl. 514/34; 514/449; 514/269; 514/49; 514/492

(57) ABSTRACT

A treatment, method and composition is provided for combating tumors by administering to a person suffering from tumors to the body tissue an effective amount of anti-cancer drugs such as 5-fluorouracil or a precursor thereof, followed by application of uracil in a pharmaceutically acceptable carrier to reduce hand-foot syndrome.

### CROSS-REFERENCES

Benefit is claimed from a Provisional Application Serial No. 60/355,764, filed Feb. 12, 2002.

### BACKGROUND OF THE INVENTION

1. Field of the Invention

The invention concerns reducing side-effects of certain chemotherapy, and particularly, side-effects manifested topically in skin redness and cracking.

2. The Related Art

An important current chemotherapy is the use of 5-fluorouracil and of its precursors such as Xeloda. Chemically, fluorouracil is 5-fluoro-2,4 (1H, 3H)-pyrimidinedione.

There is evidence that the metabolism of fluorouracil in the anabolic pathway blocks the methylation reaction of deoxyuridylic acid to thymidylic acid. In this manner, fluorouracil interferes with the synthesis of DNA and to a lesser extent, inhibits the formation of RNA. Since DNA and RNA are essential for cell division and growth, the effect of fluorouracil is believed to create a thymine deficiency which provokes unbalanced growth and death of the cell. The effects of DNA and RNA deprivation are most marked on those cells which grow more rapidly and take in fluorouracil at a more rapid rate. The catabolic metabolism of fluorouracil results in degradation products, such as urea and alpha-fluoro-beta-alanine. Among those adverse reactions to the treatment include pain, pruritus, burning, irritation, inflammation, allergic contact dermatitis, and telangiectasia. Occasionally, hyperpigmentation and scarring have also been reported.

Among all the contra indications of the treatment, there has especially been identified the significant toxic effect known as “hand-foot” syndrome. “Hand-foot” syndrome is characterized by progressive skin redness and skin cracking. The syndrome can limit the dosage amount of fluorouracil or precursors administered to a patient. Currently, “hand-foot” syndrome is dealt with by a dosage reduction and this can result in the risk of reduced efficacy against tumors.

Previous work suggests that the administration of uracil, together with a precursor of 5-fluorouracil, increases the metabolism of the latter and decreases the degradation of the former.

Compilations relevant to this field include the following.

Mackean M, Planting A, Twelves C et al: Phase 1 and pharmacologic study of intermittent twice-daily oral therapy with capecitabine in patients with advanced and/or metastatic cancer. J. Clin Oncol 16:2977-2985, 1998.

Cao S, Frank C, Shirasaka et al: 5-fluorouracil pro drug: role of anabolic and catabolic pathway modulation in therapy of colorectal cancer. Clin Can Res 1:839-845, 1995.

Hoff, P: The tegafur-based dehydropyrimidine dehydrogenase inhibitory fluoropyrimidines, UFT/eucovorin (Orzel) and S-1: a review of their clinical development and therapeutic potential. Invest New Drugs 18:331-342, 2000.

Johnson M, Hageboutros A, Wang K, et al: Life threatening toxicity in a dihydropyrimidine dehydrogenase-deficient patient after treatment with topical 5-fluorouracil. Clin Can Res 5:2006-2011, 1999.

An object of the present invention was to create a drug more toxic to tumors than 5-fluorouracil.

Another object of the present invention was to increase the efficacy of 5-fluorouracil and its precursors while reducing the side-effects associated with “hand-foot” syndrome.

### SUMMARY OF THE INVENTION

A treatment is provided against tumors which includes:

administering to a person with tumors on a body tissue in an effective amount to reduce tumor size of an anti-cancer active such as 5-fluorouracil or precursors thereof; and thereafter

applying to the body tissue an effective amount to reduce “hand-foot” syndrome of uracil in a pharmaceutically acceptable carrier.

Particularly affected by the aforementioned treatment are the hand-foot syndrome manifestations of skin redness and cracking.

Among the most preferred carriers for the uracil are such materials as fatty acids or alcohols of 8-22 carbon atoms, hydrocarbons, silicones and combinations thereof.

Furthermore, there is provided a method for reducing hand-foot syndrome associate with chemotherapy which includes applying uracil to a tumor bearing body tissue in an effective amount to reduce hand-foot syndrome.

With respect to the aforementioned method, the chemotherapy is based upon administering to the body tissue 5-fluorouracil or a precursor thereof.

Uracil may be formulated into a cream or a lotion form. Amounts of this active material may range from about 0.01 to about 60% by weight of the cream or lotion.

Yet another aspect of the present invention is provision of a composition for treating hand-foot syndrome. The composition will include about 0.1 to about 5% by weight of uracil delivered in a pharmaceutically acceptable carrier.

### DETAILED DESCRIPTION OF THE INVENTION

Use of uracil formulations can be effective as post-chemotherapy treatment providing benefit to the adverse skin effects of the chemotherapy chemicals. Among those effects that can be mitigated include redness (erythema) and cracking.

Chemotherapy agents applicable to the present treatment and methods include 5-fluorouracil or precursors thereof, thalidamide, Oxaliplatin, Xeloda, Adriamycin, Cytoxan, Taxol, Taxotere and Navelbine.

Amounts of uracil as the active formulation may range from about 0.01 to about 60%, preferably from about 0.1 to about 10%, optimally from about 1 to 5% by weight.

Formulations of uracil may be in any convenient format. These include creams, lotions, ointments, aerosol sprays, roll-on liquids, sticks and injectable forms. Besides topical applications, uracil can be administered internally through ingestion in liquid, gel cap, powder and tablet forms.

Most preferred is the topical application. Any pharmaceutically acceptable carrier may be utilized to deliver the uracil.

Treatment compositions of the present invention are particularly preferred when in the anhydrous form (less than about 5% water, preferably less than about 1% water). Yet oil and water emulsions may also be suitable for the present invention. Whether anhydrous or emulsion type, compositions of the present invention may further include a variety of pharmaceutically acceptable carriers and skin actives. Amounts of the carrier may range from about 1 to about 99%, preferably from about 5 to about 70%, optimally from about 10 to about 40% by weight. Among the carriers are emollients, water, inorganic powders, foaming agents, emulsifiers and combinations thereof.

Emollients are substances selected from polyols, esters and hydrocarbons. Polyols suitable for the invention may include propylene glycol, dipropylene glycol, polypropylene glycol, polyethylene glycol, sorbitol, hydroxypropyl sorbitol, hexylene glycol, 1,3-butylene glycol, 1,2.6-hexanetriol, glycerin, ethoxylated glycerin, propoxylated glycerin, xylitol and mixtures thereof.

Esters useful as emollients include:

(1) Alkyl esters of fatty acids having 10 to 20 carbon atoms. Methyl, isopropyl, and butyl esters of fatty acids are useful herein. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. Particularly preferred are C12-C15 alcohol benzoate esters.

(2) Alkenyl esters of fatty acids having 10 to 20 carbon atoms. Examples thereof include oleyl myristate, oleyl stearate and oleyl oleate.

(3) Ether-esters such as fatty acids esters of ethoxylated fatty alcohols.

(4) Polyhydric alcohol esters. Ethylene glycol mono and di-fatty acid esters, diethylene glycol mono- and di-fatty acid esters, polyethylene glycol (200-6000) mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.

(5) Wax esters such as beeswax, spermaceti, myristyl myristate, stearyl stearate.

(6) Sterol esters, of which cholesterol fatty acid esters are examples thereof

Illustrative hydrocarbons are mineral oil, polyalphaolefins, petrolatum, isoparaffin, polybutenes and mixtures thereof.

Inorganic powders are useful carriers. Examples include clays (such as Montmorillonite, Hectorite, Laponite and Bentonite), talc, mica, silica, alumina, zeolites, sodium sulfate, sodium bicarbonate, sodium carbonate, calcium sulfate and mixtures thereof.

Aerosol propellants may also be used as carriers. Propellants are normally based on volatile hydrocarbons such as propane, butane, isobutene, pentane, isopropane and mixtures thereof Philipps Petroleum Company is a source of such propellants under trademarks including A31, A32, A51 and A70. Halocarbons including fluorocarbons and dimethyl ether are further widely employed propellants.

Emulsifiers may constitute at least a portion of the carrier for compositions according to the present invention. These may be selected from nonionic, anionic, cationic, or amphoteric emulsifying agents. They may range in amount anywhere from about 0.1 to about 20% by weight. Illustrative nonionic emulsifiers are alkoxylated compounds based on C10-C22 fatty alcohols and acids and sorbitan. These materials are available, for instance, from the Shell Chemical Company under the Neodol trademark. Copolymers of polyoxypropylenepolyoxyethylene sold by the BASF Corporation under the Pluronic trademark are sometimes also useful. Alkyl polyglycosides available from the Henkel Corporation may also be utilized for purposes of this invention.

Anionic type emulsifiers include fatty acid soaps, sodium lauryl sulphate, sodium lauryl ether sulphate, alkyl benzene sulphonate, mono- and di-alkyl acid phosphates, sarcosinates, taurates and sodium fatty acyl isethionate.

Amphoteric emulsifiers include such materials as dialkylamine oxide and various types of betaines (such as cocamidopropyl betaine).

Except in the operating and comparative examples, or where otherwise explicitly indicated, all numbers in this description indicating amounts of material ought to be understood as modified by the word “about.”

The following examples will more fully illustrate the embodiments of this invention. All parts, percentages and proportions referred to herein and in the appended claims are by weight unless otherwise illustrated.

All documents mentioned in this application should be considered as being incorporated herein by reference.

### EXAMPLE 1

A patient being treated with orally administered Xeloda had to discontinue the routine after only 5 of 14 days of planned treatment. Discontinuance was the result of severe hand-foot syndrome. Tumor measurements on the original date and after 21 days subsequent to hand-foot syndrome recovery are recorded in Table 1 below as “initial” and “Day 21,” respectively. After two courses of treatment with Xeloda and a concurrent topical application of 1% uracil cream based on a vanishing cream base (applied four times a day to the hands and feet), the patient had no symptoms of the syndrome. See “Day 35.”

TABLE 1

| Tumor | Original Date | Day 21 | Day 35 |
|---|---|---|---|
| Breast mass | 7 × 7 cm | 9 × 9 cm | 8.5 × 8.5 cm |
| Left supraclavicular node | 1 × 1 cm | 2 × 2 cm | 1.5 × 1.5 cm |
| Right axillary mass | 2 × 2 cm | 3 × 3 cm | 2 × 2 cm |

The administration of the uracil cream both prevented the hand-foot syndrome as well as restored the anti-tumor activity of the Xeloda.

This 48 year old female patient exhibited metastatic breast cancer. She had refused mastectomy and had previously failed adriamycin and cytoxan, weekly taxol, weekly navelbine. She was then placed on Xeloda combined with use of a 1% uracil cream. Table 2 below summarizes results of this patient.

TABLE 2

| Course q3wk | 1 | 2 | 3 | 4 | 5 | 6 | 7 | 8 |
|---|---|---|---|---|---|---|---|---|
| Xeloda dose | 1250 | same | D/C | 1000 | 1250 | same | same | same |
| 14/21 days | mg/m2 | | after | mg/m2 | mg/m2 | | | |
| | bid × 14 | | 4 days | bid × 14 | bi × 14 | | | |
| Taxotere 75 mg/m2 | + | + | + | + | + | + | + | + |
| Marker tumor size | 12 × 12 | 8 × 8 | 7 × 7 | 7 × 7 | 9 × 9 | 8.5 × 8.5 | 8 × 8 | 8.5 × 8.5 |
| cm-prior to rx | | | | | | | | |
| | | | | | progression | | | |
| | | | | | on lower | | | |
| | | | | | dose Xeloda | | | |
| 1% uracil cream | 0 | 0 | 0 | 0 | + | + | + | + |
| Hand-foot syndrome | ND* | ND | ++++ | ++ | 0 | 0 | 0 | 0 |

*Not described

The 1% uracil cream allowed a reescalation of the dose of Xeloda. The results show recovered anti-tumor activity at the higher dose of Xeloda. The 1% uracil cream did not interfere with the anti-cancer activity and had no discernible toxicity.

### EXAMPLE 2

Another patient, a 68 year old white female diagnosed with metastatic colon cancer, was treated with the Xeloda and thalidomide. Hand-foot syndrome developed. Complete reversal of the syndrome occurred after topical treatment with a 1% uracil cream. The efficacy of the Xeloda and thalidomide treatment was unaffected by the uracil cream. There were no dose reductions of chemotherapy or treatment delays.

### EXAMPLE 3

A 60 year old white female with metastatic colon cancer was treated with SFU, Leucovocin, and Oxaliplatin, a very common regime against this form of cancer. Hand-foot syndrome appeared with this patient. Topical application of 1% uracil cream resulted in complete resolution of the syndrome. The anti-cancer treatment remained efficacious. No side-effects were noted as a result of the uracil cream applications. There were no dose reductions of chemotherapy or treatment delays.

The foregoing description and examples illustrate selected embodiments of the present invention. In light thereof, variations and modifications will be suggested to one skilled in the art, all of which are within the spirit and purview of this invention.

What is claimed is:

1. A treatment against tumors comprising:
administering to a person with tumor on a body tissue an effective amount to reduce tumor size of an anti-cancer active material; and thereafter
applying to the body tissue an effective amount of uracil to reduce hand-foot syndrome in a pharmaceutically acceptable carrier.

2. The treatment according to claim 1 wherein the anti-cancer active material is selected from the group consisting of 5-fluorouracil or a precursor thereof, Adriamycin, Cytoxan, Taxol, Taxotere, Navelbine, Oxaliplatin and mixtures thereof.

3. The treatment according to claim 2 wherein the active is 5-fluorouracil or a precursor thereof.

4. The treatment according to claim 1 wherein the hand-foot syndrome comprises skin redness and cracking.

5. The treatment according to claim 1 wherein the pharmaceutically acceptable carrier is selected from the group consisting of emollients, water, inorganic powders, foaming agents, emulsifiers and combinations thereof.

6. The treatment according to claim 1 wherein the pharmaceutically acceptable carrier is a fatty acid or alcohol of from 8 to 22 carbon atoms, a hydrocarbon, a silicone and combinations thereof.

7. A method for reducing hand-foot syndrome associated with chemotherapy comprising applying uracil to a tumor bearing body tissue in an effective amount to reduce the hand-foot syndrome.

8. The method according to claim 7 wherein the chemotherapy is based upon administration to the body tissue of an active selected from the group consisting of 5-fluorouracil or a precursor thereof, Adriamycin, Cytoxan, Taxol, Taxotere, Navelbine, Oxaliplatin and combinations thereof.

9. The method according to claim 8 wherein the active is 5-fluorouracil or a precursor thereof.

10. The method according to claim 7 wherein uracil is delivered in a pharmaceutically acceptable carrier.

11. The method according to claim 7 wherein uracil is formulated into a cream or lotion in a concentration from about 0.01 to about 60% by weight.

12. A composition for treating hand-foot syndrome comprising from about 0.01 to about 60% uracil in a pharmaceutically acceptable carrier.
